(19) United States
(12) Patent Application Publication
(10) Pub. No.: US 2025/0332019 A1
(43) Pub. Date: Oct. 30, 2025

(54) HAND-OPERABLE ANTEGRADE OSTOMY INFUSION DEVICE

(71) Applicant: The Regents of the University of California, Oakland, CA (US)

(72) Inventors: Shanglei Liu, San Diego, CA (US); Julian Fricks, La Jolla, CA (US)

(21) Appl. No.: 19/190,500

(22) Filed: Apr. 25, 2025

Related U.S. Application Data
(60) Provisional application No. 63/638,877, filed on Apr. 25, 2024.

(51) Int. Cl. A61F 5/44; A61M 37/00; A61M 39/24

(52) U.S. Cl. CPC A61F 5/4408; A61M 37/00; A61M 39/24; A61M 2202/0021; A61M 2205/0216; A61M 2205/3331

(57) ABSTRACT

Aspects of this technical solution can include an assembly including an ostomy bag and an antegrade ostomy infusion device that is fluidly coupled to the ostomy bag. The antegrade ostomy infusion device includes a deformable reservoir that is configured to deliver a fluid from the ostomy bag to a patient's distal stoma.

### CROSS-REFERENCE TO RELATED APPLICATIONS

This application claims the benefit of and priority to U.S. Provisional Patent Application No. 63/638,877, filed Apr. 25, 2024, the entire contents of which are hereby incorporated by reference herein.

### TECHNICAL FIELD

The present implementations relate generally to medical devices, including but not limited to antegrade ostomy infusion devices.

### SUMMARY

Ileostomies can be performed on patients in gastrointestinal (GI) surgery for indications such as protecting a distal anastomosis or to create a route for waste evacuation. Ileostomy takedown procedures can be performed to reverse an ileostomy. However, Ileostomy takedown procedures can have complications such as postoperative ileus in as high as 37% of cases as well as other complications including anastomosis leak, colitis related to long periods of disuse, incontinence, intestinal deconditioning, and others. Such complications can severely impact the patient's postoperative quality of life, increase postoperative length of stay, and increase frequency of hospital readmission. A significant decrease in the incidence of these adverse effects can be achieved where the patient's own chyme is administered to the distal stoma in the days or weeks prior to the takedown procedure.

This technical solution is directed at least to an affordable, manually operated (e.g., patient operated, hand-operable, etc.) pump that deliver ostomy bag contents into the distal intestine. In some embodiments, the device is housed within an ostomy bag, which can avoid messy spillage and simplify device operation. The device can be used by patients, physicians, nurses, and caretakers to help prepare a patient for ileostomy takedown and improve postoperative outcomes by reintroduction of intestine contents to the diverted intestine prior to committing to the risks of an Ileostomy takedown, which is a major operation. Thus, a technical solution for a manually-operable (e.g., hand-operable) antegrade ostomy infusion device is provided.

At least one aspect is directed to a device. The device can include a deformable reservoir (e.g., a bulb). The device can include a catheter having an end coupled with the deformable reservoir. In some embodiments, the catheter is a melacot catheter or another form of blunt tipped soft catheter. The device can include an ostomy bag at least partially enclosing the bulb and the catheter.

In some embodiments, the device also includes two one-way valves, including a first one-way valve disposed at an inflow end of the reservoir, and a second one-way valve disposed at an outlet end of the reservoir at which the reservoir is fluidly coupled to the catheter, such that intestinal contents can be brought into the reservoir (e.g., from the ostomy bag) in a unidirectional manner. During use, as the reservoir is compressed and returns to its original shape elastically, the reservoir generates a negative pressure that fills the reservoir (e.g., through the first one-way valve). During a subsequent compression of the reservoir, the contents inside the reservoir flow to the catheter (e.g., through the second one-way valve), and as the reservoir again returns to its original shape, will refill the reservoir (e.g., from the first one-way valve) as before.

At least one aspect is directed to a method. The method can include deforming a reservoir coupled with a first end of a catheter. The method can include transporting, in response to the deforming, a fluid from the reservoir to a second end of the catheter.

In some embodiments, the method includes transporting the fluid through two one-way valves to create unidirectional flow through the reservoir and the catheter, directing the outflow from the catheter that is inserted into the patient's intestine externally through the existing efferent limb of the ileostomy.

At least one aspect relates to an assembly including an ostomy bag and an antegrade ostomy infusion device that is fluidly coupled to the ostomy bag. The antegrade ostomy infusion device includes a deformable reservoir that is configured to deliver a fluid from the ostomy bag to a patient's distal stoma.

At least one aspect relates to a device including a deformable reservoir, a catheter, a first valve, and a second valve. The catheter has a first end coupled with the deformable reservoir. The first valve fluidly couples the deformable reservoir to the catheter. The second valve is coupled to the deformable reservoir at an inlet of the deformable reservoir.

At least one aspect relates to a method including deforming a reservoir coupled to an ostomy bag at a first end of the reservoir to draw a fluid into the reservoir from the ostomy bag; deforming the reservoir after receiving fluid from the ostomy bag to transport the fluid from the reservoir through a catheter that is coupled to the reservoir distal from the first end.

### BRIEF DESCRIPTION OF THE FIGURES

These and other aspects and features of the present implementations are depicted by way of example in the figures discussed herein. Present implementations can be directed to, but are not limited to, examples depicted in the figures discussed herein. Thus, this disclosure is not limited to any figure or portion thereof depicted or referenced herein, or any aspect described herein with respect to any figures depicted or referenced herein.

FIG. 1 depicts an example device, in accordance with present implementations.

FIG. 2 depicts an example device, in accordance with present implementations.

FIG. 3 depicts an example environment, in accordance with present implementations.

FIG. 4 depicts an example device, in accordance with present implementations.

FIG. 5 depicts an example method of operating a hand-operable antegrade ostomy infusion device, in accordance with present implementations.

FIG. 6 depicts an example method of operating a hand-operable antegrade ostomy infusion device, in accordance with present implementations.

### DETAILED DESCRIPTION

Aspects of this technical solution are described herein with reference to the figures, which are illustrative examples of this technical solution. The figures and examples below are not meant to limit the scope of this technical solution to the present implementations or to a single implementation, and other implementations in accordance with present implementations are possible, for example, by way of interchange of some or all of the described or illustrated elements. Where certain elements of the present implementations can be partially or fully implemented using known components, only those portions of such known components that are necessary for an understanding of the present implementations are described, and detailed descriptions of other portions of such known components are omitted to not obscure the present implementations. Terms in the specification and claims are to be ascribed no uncommon or special meaning unless explicitly set forth herein. Further, this technical solution and the present implementations encompass present and future known equivalents to the known components referred to herein by way of description, illustration, or example.

Aspects herein relate to a manually-operable (e.g., hand-operable) antegrade ostomy infusion device. The ostomy infusion device can enable fluid transport capability of fluids from within an ostomy bag to reintroduce a patient's own chyme into the patient's distal stoma. In various embodiments, the device includes a deformable fluid reservoir, which can reduce complexity relative to electronically operated pumps to reduce expense and difficulty of operation by and for patients. This technical solution can provide at least a technical improvement of eliminating consistent upkeep, charging, and maintenance. Thus, the technical solution includes a simpler and more robust design. Interaction with the patient's small intestine informs material choices that will minimize stress on the small bowel.

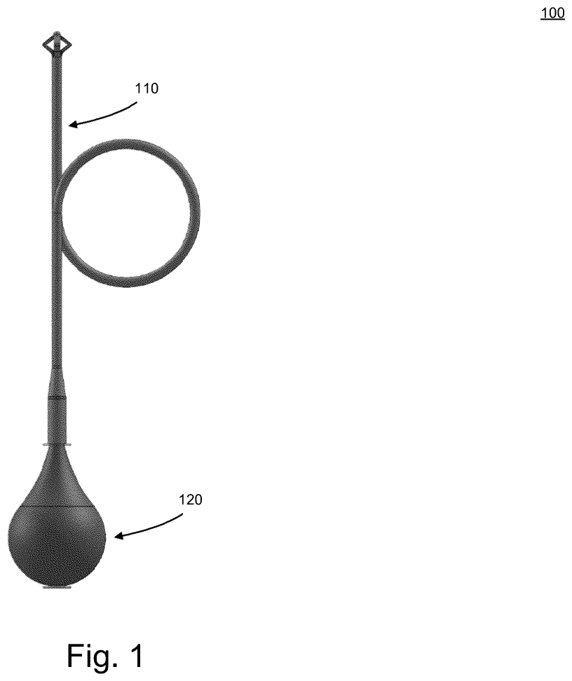

FIG. 1 depicts an example device, in accordance with present implementations. As illustrated by way of example in FIG. 1, an example device 100 (which can also be referred to as an antegrade ostomy infusion system, an antegrade ostomy infusion assembly, and/or an antegrade ostomy infusion device) can include at least a catheter 110 and a deformable reservoir, shown as reservoir 120. The reservoir 120 120 is connected to the catheter 110.

The reservoir 120 is configured to deliver a fluid from an ostomy bag to a patient's distal stoma. In some embodiments, the reservoir 120 is a suction bulb that is made from rubber, silicon, or another elastically deformable material. The reservoir 120 includes a bulb 122 and a tip 124 that extends from the bulb 122 and that fluidly couples the bulb 122 to the catheter 110. The bulb 122 is deformable between a compressed position and an expanded position having a greater internal volume as compared to the compressed position (e.g., a greater fluid receiving volume as compared to the compressed position). The bulb 122 is made from a deformable elastic material that is configured to return to the expanded position on its own after being compressed.

The catheter 110 includes a conduit that is fluidly coupled to the reservoir 120. In some embodiments, as shown in FIG. 1, the catheter 110 is a malecot catheter that includes a conduit and malecot wings or protrusions at an end (e.g., a second end, distal to the first end that is attached to the reservoir 120) of the conduit opposite the reservoir 120, which protrude radially away from a central axis of the catheter 110. The wings can prevent blockage and promote retention of the catheter 110 in the patient during use. In other embodiments, the catheter 110 may be another type of blunt soft tipped catheter, or another type of catheter.

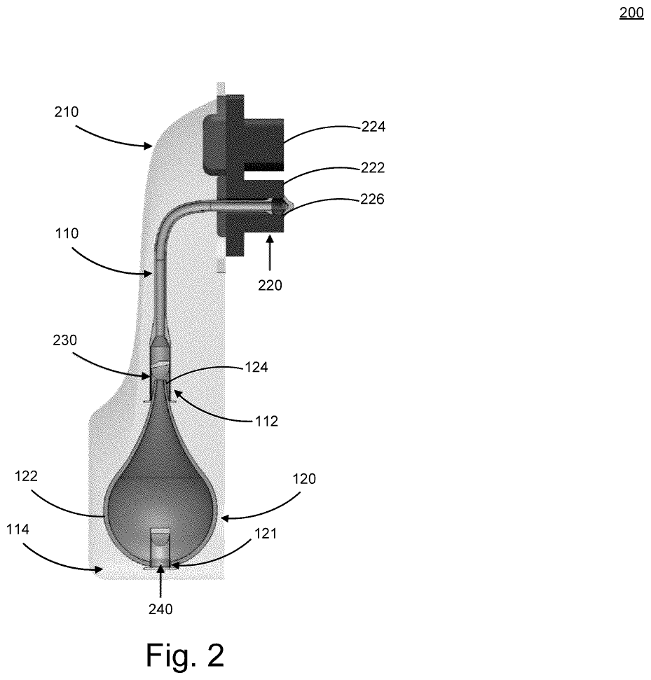

FIG. 2 depicts an example device that includes the catheter 110 of FIG. 1, in accordance with present implementations. As illustrated by way of example in FIG. 2, an example device 200 (which can also be referred to as an antegrade ostomy infusion system, an antegrade ostomy infusion assembly, and/or an antegrade ostomy infusion device) can include, in addition to components of the catheter 110 in FIG. 1, an ostomy bag 210, a distal stoma of ileostomy 220, a first valve 230, and a second valve 240. The device 200 can correspond at least partially in one or more of structure and operation to the device 100 and can include one or more portions or parts of the device 100.

In the embodiment of FIG. 2, the ostomy bag 210 at least partially encloses the reservoir 120. The device 100 is disposed within the ostomy bag 210 such that the reservoir 120 is manually compressible responsive to an external force applied to an outer surface of the ostomy bag 210. Such an arrangement can simplify manufacturing and reduce the risk of leakage during use. In other embodiments, at least portions of the device 100 may be disposed outside of the ostomy bag 210. For example, the reservoir 120 may be fluidly coupled to the ostomy bag 210 at a lower end thereof, such as by a one-way check valve (as will be further described), to selectively fluidly couple the reservoir 120 to the ostomy bag 210 responsive to negative pressure within the reservoir 120.

The device 100 can include a rubber suction reservoir, shown as reservoir 120, connected to a catheter 110, with two valves, shown as a first valve 230 and a second valve 240, situated proximate to, or at, a proximal end (e.g., a first end), and a distal end (e.g., a second end) of the reservoir 120. In the embodiment of FIG. 2, the first valve 230 is disposed within a first end 112 of the catheter 110 at a fluid connection between the tip 124 of the reservoir 120 and the catheter 110. In other embodiments, the first valve 230 may be directly coupled to the tip 124 or to the reservoir 120 upstream from the tip 124. In some embodiments, the first valve 230 is a one-way valve that is configured to selectively couple the reservoir 120 to the ostomy bag 210. The first one-way valve is configured to provide unidirectional flow from the reservoir 120 (e.g., an inner cavity defined by an outer wall of the reservoir 120) into the catheter 110, and to substantially prevent or impair backflow from the catheter 110 back into the reservoir 120.

The second valve 240 is disposed at the proximal end of the reservoir 120 (e.g., at a lower end of the bulb 122 as shown in FIG. 2), at an inlet 121 to the reservoir 120, opposite from the tip 124. In some embodiments, the second valve 240 is a second one-way valve that is configured to selectively fluidly couple the reservoir 120 to the catheter 110. The second valve 240 is configured to provide unidirectional flow from the cavity of the ostomy bag 210 into the reservoir 120, and to substantially prevent or impair backflow from the reservoir 120 back into the ostomy bag 210.

In some embodiments, the first valve 230 and the second valve 240 are diaphragm check valves that each include a flexible diaphragm and configured to open with forward flow and seal against reverse flow. In other embodiments, the first valve 230 and the second valve 240 may be or include another type of check valve that may be the same or different from one another. In some embodiments, the first valve 230 and the second valve 240 are sealed and secured to one of the reservoir 120 and the catheter 110 with medical grade adhesive. The ostomy bag 210 defines a cavity that is sized to receive the reservoir 120 and the catheter 110 therein. As the device is meant to sit in the ostomy bag 210, the reservoir 120 can be operated through it.

For example, as shown in FIG. 2, the device 200 can include the first valve 230 disposed at a first end of the reservoir 120. For example, the first valve 230 is configured to impair movement of fluid from the catheter 110 into the reservoir 120. For example, the second valve 240 is disposed at a second end of the reservoir 120 (e.g., proximate to the tip 124), the second end of the reservoir 120 distal to the first end of the reservoir 120. For example, the second valve 240 is configured to impair movement of a fluid 114 from the reservoir 120 into the ostomy bag 210.

In some embodiments, as shown in FIG. 2, a distal end (e.g., a second end) of the catheter 110 is fluidly coupled to the distal stoma of ileostomy 220. The distal stoma of ileostomy 220 may include a connector 222, port, tissue, or another access to the patient's distal stoma. The connector 222 may also be configured to fluidly couple the ostomy bag 210 to a patient. In some embodiments, the connector 222 defines multiple access openings, including a first opening 224 that is configured to direct waste from the patient into the ostomy bag 210, and a second opening 226 offset from the first opening 224 that is configured to fluidly couple the catheter 110 to a patient's distal stoma. In some embodiments, the device 100, including the reservoir 120, the catheter 110, the first valve 230, and/or the second valve 240 are integrally formed from a single piece of materials from an injection process. In other embodiments, at least a portion of the device 100 is manufactured separately from other components. For example, the reservoir 120 and the catheter 110 may be integrally formed as a unitary body and at least one of the first valve 230 and the second valve 240 are connected thereto via a medical grade silicone adhesive, or by another form of leak-tight connection.

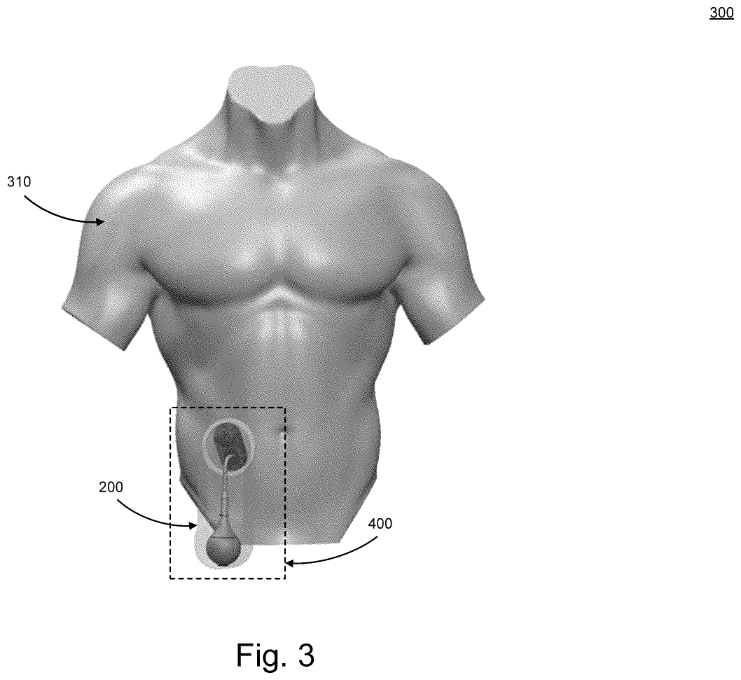

FIG. 3 depicts an example environment, in accordance with present implementations. As illustrated by way of example in FIG. 3, an example environment 300 can include at least a patient 310. The patient 310 can befitted with the device 100 or 200.

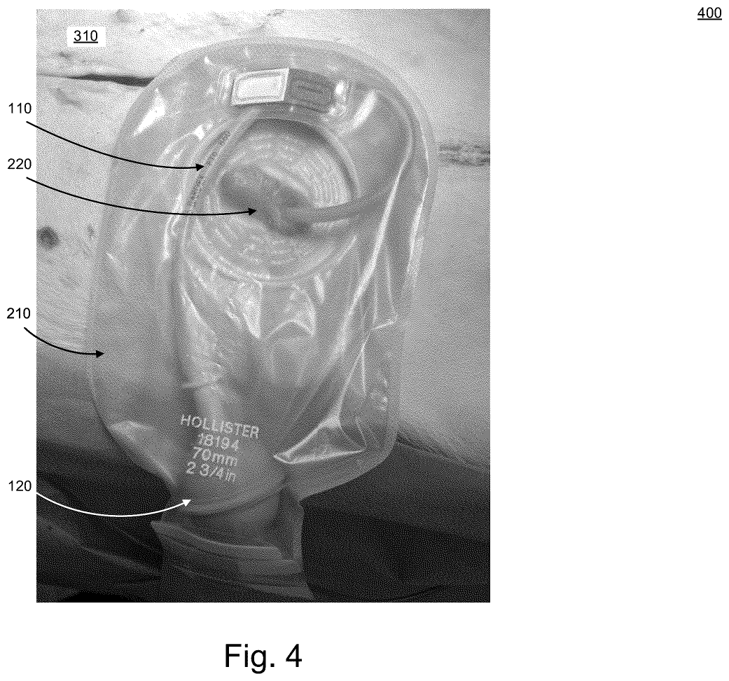

FIG. 4 depicts an example device, in accordance with present implementations. As illustrated by way of example in FIG. 4, an example device 400 can correspond at least partially in one or more of structure and operation to one or more of the device 100 and 200.

The reservoir (e.g., the reservoir 120 of FIGS. 1-2) is configured so that squeezing the bulb (e.g., the bulb 122) pushes contents out of the tip (e.g., the distal end) of the catheter (e.g., the catheter 110), and releasing the bulb creates negative pressure that sucks contents into the bulb. The device is configured such that squeezing the bulb will pump contents of the ostomy bag into the distal stoma. This can have a therapeutic effect as described previously. The device itself has already demonstrated mechanical functionality in multiple prototypes.

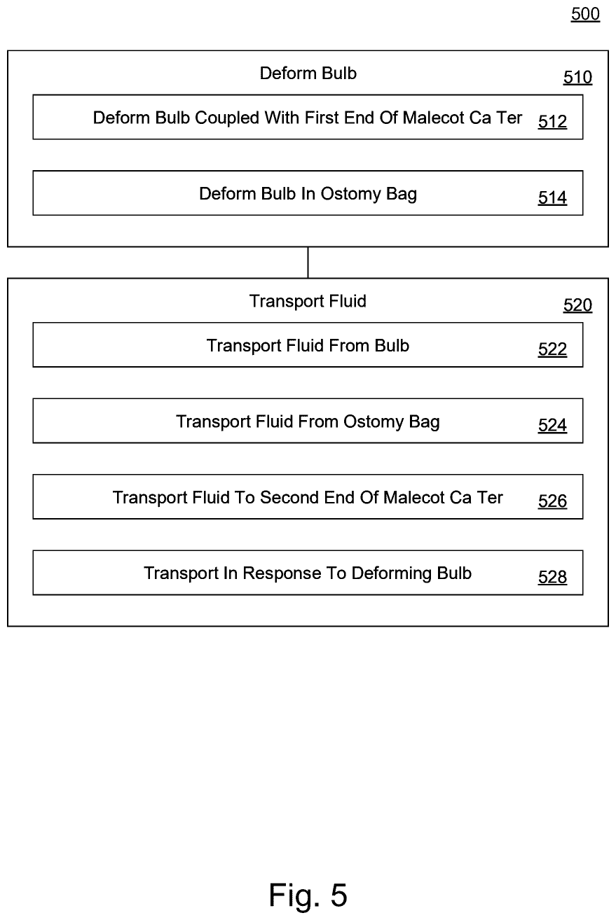

FIG. 5 depicts an example method of operating a manually-operable (e.g., a hand-operable) antegrade ostomy infusion device, in accordance with present implementations. At least the devices 100, 200, or 400 can perform method 500.

At 510, the method 500 can include deforming a reservoir (e.g., a bulb). At 512, the method 500 can include deforming a reservoir coupled with a first end of a catheter. At 514, the method 500 can include deforming the reservoir (e.g., a bulb of the reservoir) by depressing an outer surface of an ostomy bag. The method 500 can include deforming the reservoir coupled to the ostomy bag at a first end of the reservoir to draw a fluid into the reservoir from the ostomy bag.

At 520, the method 500 can include transporting a fluid, such as by deforming the reservoir after receiving fluid from the ostomy bag to transport the fluid from the reservoir through a catheter that is coupled to the reservoir distal from the first end.

At 522, the method 500 can include transporting a fluid from the bulb. For example, operation 522 can include transporting the fluid from the reservoir through a first one-way valve that is configured to impair movement of the fluid into the reservoir from the catheter.

At 524, the method 500 can transport a fluid from an ostomy bag. For example, operation 524 can include drawing fluid into the reservoir through a second one-way valve disposed at the first end of the reservoir and that is configured to impair movement of fluid from the catheter to the reservoir.

At 526, the method 500 can include transporting a fluid to a second end of the catheter, such as responsive to depressing the reservoir after receiving fluid from the ostomy bag. At 528, the method 500 can include transporting the fluid to a patient's distal stoma (e.g., through a connector configured to fluidly couple the ostomy bag to a patient) in response to the deforming reservoir.

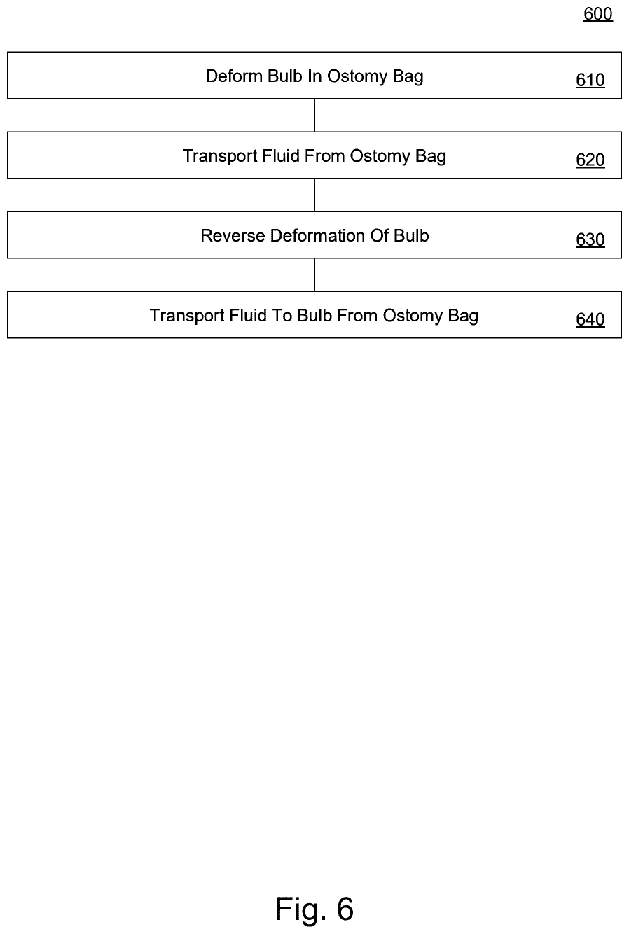

FIG. 6 depicts an example method of operating a manually-operable (e.g., hand-operable) antegrade ostomy infusion device, in accordance with present implementations. At least the devices 100, 200, or 400 can perform method 600. At 610, the method 600 can include deforming a reservoir that is disposed at least partially within an ostomy bag.

At 620, the method 600 can include transporting a fluid from the ostomy bag. For example, operation 620 can include transporting, in response to the deforming and via a first valve configured to impair movement of fluid from the catheter to the reservoir, the fluid.

At 630, the method 600 can include reversing deformation of the reservoir. For example, operation 630 can include reversing at least partially the deforming the reservoir to transport fluid to the reservoir from an ostomy bag coupled with the reservoir.

At 640, the method 600 can include transporting a fluid to the reservoir from the ostomy bag. For example, operation 640 can include transporting, in response to the reversing and via a second valve configured to impair movement of fluid from the reservoir to the ostomy bag, the fluid.

A method of making a manually-operable (e.g., a hand-operable) antegrade ostomy infusion assembly can include enclosing an antegrade ostomy infusion device at least partially within an ostomy bag. Such an implementation may include inserting the infusion device, including a deformable reservoir and a catheter, into the ostomy bag through an opening in the ostomy bag. The method can also include connecting a first end of the catheter to the reservoir and connecting a second end of the catheter that is distal from the first end to a connector. The connector may beat least partially disposed within the opening. In some embodiments, the method can include fluidly coupling the catheter to the reservoir by a first one-way valve and fluidly coupling the reservoir to an interior cavity of the ostomy bag by a second one-way valve that is separate from the first one-way valve.

Having now described some illustrative implementations, the foregoing is illustrative and not limiting, having been presented by way of example. In particular, although many of the examples presented herein involve specific combinations of method acts or system elements, those acts and those elements may be combined in other was to accomplish the same objectives. Acts, elements, and features discussed in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both “A’ and ‘B’. Such references used in conjunction with “comprising” or other open terminology can include additional items. References to “is” or “are” may be construed as nonlimiting to the implementation or action referenced in connection with that term. The terms “is” or “are” or any tense or derivative thereof, are interchangeable and synonymous with “can be” as used herein, unless stated otherwise herein.

Directional indicators depicted herein are example directions to facilitate understanding of the examples discussed herein, and are not limited to the directional indicators depicted herein. Any directional indicator depicted herein can be modified to the reverse direction, or can be modified to include both the depicted direction and a direction reverse to the depicted direction, unless stated otherwise herein. While operations are depicted in the drawings in a particular order, such operations are not required to be performed in the particular order shown or in sequential order, and all illustrated operations are not required to be performed. Actions described herein can be performed in a different order. Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any clam elements.

Scope of the systems and methods described herein is thus indicated by the appended claims, rather than the foregoing description. The scope of the claims includes equivalents to the meaning and scope of the appended claims.

What is claimed is:

1. An assembly, comprising:
an ostomy bag; and
an antegrade ostomy infusion device fluidly coupled to the ostomy bag, the antegrade ostomy infusion device comprising a deformable reservoir that is configured to deliver a fluid from the ostomy bag to a patient's distal stoma.

2. The assembly of claim 1, wherein the antegrade ostomy infusion device is disposed within the ostomy bag.

3. The assembly of claim 1, wherein the antegrade ostomy infusion device further comprises a catheter having a first end coupled to the deformable reservoir.

4. The assembly of claim 1, further comprising:
a first valve coupled to the deformable reservoir and configured to selectively fluidly couple the deformable reservoir to the ostomy bag; and
a second valve configured to selectively fluidly couple the deformable reservoir to a catheter.

5. The assembly of claim 4, wherein the first valve comprises a first one-way valve configured to impair movement of fluid into the deformable reservoir, and the second valve comprises a second one-way valve configured to impair movement of fluid out from the deformable reservoir.

6. The assembly of claim 1, wherein the deformable reservoir is manually compressible responsive to an external force applied to an outer surface of the ostomy bag.

7. The assembly of claim 1, wherein the ostomy bag comprises a connector that is configured to fluidly couple the ostomy bag to a patient, and further comprising a catheter that fluidly couples the deformable reservoir to the connector.

8. A device, comprising:
a deformable reservoir;
a catheter having a first end coupled with the deformable reservoir;
a first valve fluidly coupling the deformable reservoir to the catheter; and
a second valve coupled to the deformable reservoir at an inlet of the deformable reservoir.

9. The device of claim 8, further comprising an ostomy bag at least partially enclosing the deformable reservoir and the catheter.

10. The device of claim 9, wherein the ostomy bag comprises a connector that is configured to fluidly couple the ostomy bag to a patient, the catheter fluidly coupled to the connector.

11. The device of claim 10, wherein the connector defines a plurality of access openings comprising:
a first access opening configured to direct waste from the patient into the ostomy bag; and
a second opening offset from the first access opening and configured to fluidly couple the catheter to a patient's distal stoma.

12. The device of claim 9, wherein the deformable reservoir is manually compressible responsive to an external force applied to an outer surface of the ostomy bag.

13. The device of claim 8, wherein the first valve is disposed at a first end of the deformable reservoir, and the second valve is disposed at a second end of the deformable reservoir distal to the first end of the deformable reservoir.

14. The device of claim 8, wherein the deformable reservoir comprises a bulb and a tip extending from the bulb, the first valve disposed downstream from the tip.

15. The device of claim 8, wherein the first valve comprises a first one-way valve configured to impair movement of fluid from the catheter into the deformable reservoir.

16. The device of claim 15, wherein the second valve comprises a second one-way valve configured to impair movement of fluid from the deformable reservoir to an ostomy bag.

17. The device of claim 8, wherein the deformable reservoir comprises a deformable elastic material movable between a compressed position and an expanded position having a greater internal volume than the compressed position, the deformable elastic material configured to return to the expanded position on its own after being compressed.

18. A method, comprising:
deforming a reservoir coupled to an ostomy bag at a first end of the reservoir to draw a fluid into the reservoir from the ostomy bag; and
deforming the reservoir after receiving fluid from the ostomy bag to transport the fluid from the reservoir through a catheter that is coupled to the reservoir distal from the first end.

19. The method of claim 18, wherein deforming the reservoir after receiving fluid from the ostomy bag comprises transporting the fluid from the reservoir through a first one-way valve that is configured to impair movement of the fluid into the reservoir from the catheter.

20. The method of claim 18, wherein deforming the reservoir to draw the fluid into the reservoir comprises drawing fluid into the reservoir through a second one-way valve disposed at the first end of the reservoir that is configured to impair movement of fluid from the catheter to the reservoir.
